(19) United States
(12) Patent Application Publication
(10) Pub. No.: US 2020/0377450 A1
(43) Pub. Date: Dec. 3, 2020

(54) Surfactants From Sulfonation Of Aromatic Ethers

(71) Applicant: ExxonMobil Research and Engineering Company, Annandale, NJ (US)

(72) Inventors: Abhimanyu O. Patil, Westfield, NJ (US); Satish Bodige, Wayne, NJ (US); Aruna Mohan, Annandale, NJ (US); Arben Jusufi, Belle Mead, NJ (US); Kanmi Mao, Basking Ridge, NJ (US)

(21) Appl. No.: 16/859,274

(22) Filed: Apr. 27, 2020

Related U.S. Application Data
(60) Provisional application No. 62/853,426, filed on May 28, 2019.

(51) Int. Cl. C07C 309/42; C10M 135/10

(52) U.S. Cl. CPC C07C 309/42; C10M 135/10

(57) ABSTRACT

The present disclosure relates to surfactants formed from sulfonation of aromatic ethers. The example surfactant composition may include a sulfonated aromatic ether. The sulfonated aromatic ether may include an aromatic ring with substituents comprising an ether group and a sulfonic acid group or a salt of the sulfonic acid group. The ether group may be represented by the following formula:

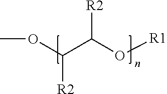

wherein R₁ is a linear or branched alkyl group having from 1 carbon atom to 20 carbon atoms; wherein each R₂ is individually a hydrogen or an alkyl group having from 1 carbon atom to 4 carbon atoms; and wherein n is a value from 0 to 8.

### FIELD

The present disclosure relates to surfactants formed from sulfonation of aromatic ethers.

### BACKGROUND

Surfactants are compounds that tend to lower the surface tension at an interface between two components. As such, surfactants may be used in a wide range of applications, which may include, for example, promoting solubility of an otherwise sparingly soluble solid, lowering viscosity of a fluid phase, and promoting foaming of a fluid. Surfactants may be found in a wide range of consumer and industrial products including, for example, soaps, detergents, cosmetics, pharmaceuticals, and dispersants.

Surfactants feature both hydrophobic and hydrophilic regions within their molecular structure. Hydrophobic regions are generally non-ionic and may include saturated or unsaturated hydrocarbyl groups, such as alkyl, alkenyl, or aryl groups. Hydrophilic regions, in contrast, may be ionic, non-ionic, or zwitterionic and encompass a range of polar functional groups or moieties. Ionic functional groups that may be present in the hydrophilic regions of various surfactants include, for example, sulfonates, sulfates, carboxylates, phosphates, quaternary ammonium groups, and the like. Non-ionic hydrophilic regions may include functional groups or moieties bearing one or more heteroatoms that are capable of receiving hydrogen bonds, such as polyethers (e.g., ethoxylates). Zwitterionic hydrophilic regions may include moieties such as betaines, sultaines, and related phospholipid compounds.

Surfactants finding extensive commercial use feature a relatively limited range of structure types. Common classes of commercial surfactants include, for example, alkylbenzene sulfonates, lignin sulfonates, long chain fatty alcohol sulfates, long chain fatty acid carboxylates, long chain fatty alcohol ethoxylates, long chain quaternary ammonium compounds, and alkylphenol ethoxylates. The various classes of surfactants may exhibit a range of surfactant properties, and there may be further property variation within the members or homologues within each class. Accordingly, a surfactant for a given application may be chosen based upon various application-specific requirements.

### SUMMARY

Disclosed herein is an example surfactant composition. The example surfactant composition may include a sulfonated aromatic ether. The sulfonated aromatic ether may include an aromatic ring with substituents comprising an ether group and a sulfonic acid group or a salt of the sulfonic acid group. The ether group may be represented by the following formula:

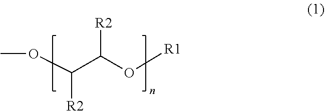

wherein R₁ is a linear or branched alkyl group having from 1 carbon atom to 20 carbon atoms, wherein each R₂ is individually a hydrogen or an alkyl group having from 1 carbon atom to 4 carbon atoms, and wherein n is a value from 0 to 8.

Further disclosed herein is an example method for producing a surfactant composition. The example method may include reacting an aromatic ether with a sulfonating reagent to form the surfactant composition, wherein the aromatic ether includes an ether group represented by Formula (I). The surfactant composition may include a sulfonated aromatic ether including an aromatic ring with substituents comprising the ether group and a sulfonic acid group.

### DETAILED DESCRIPTION

The present disclosure relates to surfactants formed from sulfonation of aromatic ethers. As discussed above, the majority of surfactants in common commercial use are based upon a relatively limited number of structural classes, including alkyl benzene sulfonates, lignin sulfonates, long chain fatty alcohol sulfonates, long chain fatty acid carboxylates, long chain fatty alcohol ethoxylates, long chain quaternary ammonium compounds, and alkylphenol ethoxylates. The various structural classes, as well as specific members or homologues within each structural class, may exhibit a range of surfactant properties, which may be chosen for suitability or compatibility with a given application. Some existing and emerging applications may have a demand for reducing the number of surfactants by increasing surfactant efficiency while also increasing the use of those that provide multiple benefits. Multifunctional surfactants that exhibit more than one property may be desirable as they can provide multiple benefits.

The present disclosure describes various surfactant compositions that include sulfonated aromatic ethers. There may be several potential advantages to the compositions and methods disclosed herein, only some of which may be alluded to in the present disclosure. One of the many potential advantages of the compositions and methods is that the surfactant compositions may be multi-functional. For example, the surfactant compositions may have conventional surfactant activity and, thus, can lower the surface tension between two components. In addition to surfactant functionality, the surfactant composition may also be used as lubricant detergents. There may be additional advantages with respect to the sulfonation reaction for formation of the surfactant compositions. Additionally, embodiments may use silica sulfuric acid as the sulfonating reagent. Silica sulfuric acid is a solid acid that is relatively stable. Because the sulfonating reaction can be carried out at moderate temperatures and the silica sulfuric acid has a high surface area, this reagent tends to react with high selectivity and the reverse reaction is low. In addition, the sulfonation reaction with the silica sulfuric acid should produce no or very small amounts of sulfones. Additional advantages from use of silica sulfuric acid are that the reaction should proceed under heterogenous conditions and also the silica sulfuric acid can be used multiple times without losing its activity.

Suitable sulfonated aromatic ethers may include an aromatic ring with substituents comprising an ether group and a sulfonic acid group, wherein the ether group is represented by Formula (1) below:

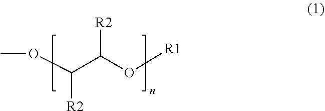

wherein R₁ is a linear or branched alkyl group having from 1 carbon atom to 20 carbon atoms, each R₂ is the same or different and is hydrogen or an alkyl group having from 1 carbon atom to 4 carbon atoms, and n is a value from 0 to 8. Illustrative R₁ substituents include, for example, linear or branched alkyl groups having from 1 carbon atom to 20 carbon atoms, linear or branched alkyl groups having from 1 carbon atom to 10 carbon atoms, or linear or branched alkyl groups having from 1 to 8 carbon atoms. Illustrative R₁ substituents include, for example, methyl, ethyl, propyl, butyl, pentyl, hexyl, heptyl, octyl, and the like. Illustrative R₂ substituents include, for example, hydrogen and alkyl groups having from 1 carbon atom to 4 carbon atoms (e.g., methyl, ethyl, propyl, butyl, and the like). Values of n can range from 0 to 8, from 0 to 6, or from 0 to 4.

The aromatic ring may be a five- or six-membered monocyclic ring or a ten- or twelve-membered bicyclic ring (e.g., naphthalene, biphenyl, cyclohexylphenyl, etc.). In some embodiments, the aromatic ring may heterocyclic or carbocyclic. Additional substituents may also be present on the aromatic ring of the sulfonated aromatic ether, including, but not limited to, an alkyl group, an alkenyl group, a heteroatom-substituted alkyl group, a hetero-atom substituted alkenyl group, or a heteroatom, such as F, Cl, or Br. The alkyl or alkenyl (or heteroatom substituted) groups may be the same or different and, in some embodiments, may include 1 carbon atom to 20 carbon atoms, from 1 carbon atom to 10 carbon atoms, from 1 carbon atom to 8 carbon atoms, or from 1 carbon atom to 4 carbon atoms. For example, suitable additional substituents may include methyl, ethyl, ethyl, propyl, butyl, pentyl, hexyl, heptyl, octyl, and the like.

In at least one embodiment, the aromatic ring may be substituted with more than one ether group of Formula (I). The ether groups may be positioned on the aromatic ring at any suitable position. For example, the aromatic ring may have an ether group in the 1-position and one or more additional ether groups at the 2-position, 3-position, 4-position, 5-position and/or 6-position. By way of further example, the aromatic ring may have ether groups in the 1-position and the 2-position. The ether groups on the aromatic ring may be the same or different.

In at least one embodiment, the aromatic ring may be substituted with one or more sulfonic acid groups or salts of the sulfonic acid group. Suitable salts may include, for example, sodium salts of the sulfonic acid groups. Other potential salts may also include ammonium salts, potassium salts, barium salts, or lithium salts. For example, the aromatic ring may include an ether group in the 1-position and one or more sulfonic groups (or salts of the sulfonic acid groups) in the 2-position, 3-position, 4-position, and/or 5-position. In some embodiments, the aromatic ring may include either groups in the 4-position and the 5-position. A specific example of a suitable sulfonated aromatic ether may include a 6-membered carbocyclic ring with ether groups in the 1-position and 2-position and a sulfonic acid group (or salt of the sulfonic acid group) in the 5-position. Another specific example of a suitable sulfonated aromatic ether may include a 6-membered carbocyclic ring with ether groups in the 1-position and 2-position and sulfonic acid groups (or salts of the sulfonic acid groups) in the 4-position and the 5-position.

An example of a suitable surfactant composition may include, but is not limited to, a sulfonated aromatic ether of Formula (2) as follows:

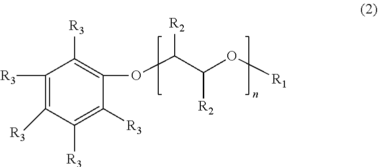

wherein R₁ is a linear or branched alkyl group having from 1 carbon atom to 20 carbon atoms, each R₂ is the same or different and is hydrogen or an alkyl group having from 1 carbon atom to 4 carbon atoms, each R₃ is a hydrogen, a sulfonic acid group, a salt of the sulfonic acid group, an ether group of Formula (I), an alkyl group, an alkenyl group, a heteroatom-substituted alkyl group, a hetero-atom substituted alkenyl group, or a heteroatom, such as F, Cl, or Br, and each n is a value from 0 to 8, wherein at least one R₃ is a sulfonic acid group. The alkyl or alkenyl (or heteroatom substituted) groups may be the same or different and, in some embodiments, may include 1 carbon atom to 20 carbon atoms, from 1 carbon atom to 10 carbon atoms, from 1 carbon atom to 8 carbon atoms, or from 1 carbon atom to 4 carbon atoms.

An additional example of a suitable surfactant composition may include, but is not limited to, a sulfonated aromatic ether of Formula (3) as follows:

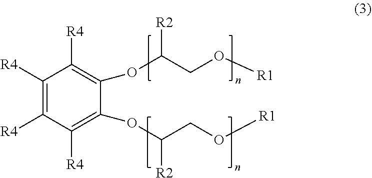

wherein R₁ is a linear or branched alkyl group having from 1 carbon atom to 20 carbon atoms, each R₂ is the same or different and is hydrogen or an alkyl group having from 1 carbon atom to 4 carbon atoms, each R₄ is a hydrogen, a sulfonic acid group, or a salt of the sulfonic acid group, and each n is a value from 0 to 8, wherein at least one R₄ is a sulfonic acid group or salt of the sulfonic acid group. The alkyl or alkenyl (or heteroatom substituted) groups may be the same or different and, in some embodiments, may include 1 carbon atom to 20 carbon atoms, from 1 carbon atom to 10 carbon atoms, from 1 carbon atom to 8 carbon atoms, or from 1 carbon atom to 4 carbon atoms.

Examples of suitable surfactant compositions of Formula (3) includes those represented by Formulae (4) to (11) further having at least one sulfonic acid group (or salt of the sulfonic acid group) at any one of the 3-position, the 4-position, 5-position and/or 6-position:

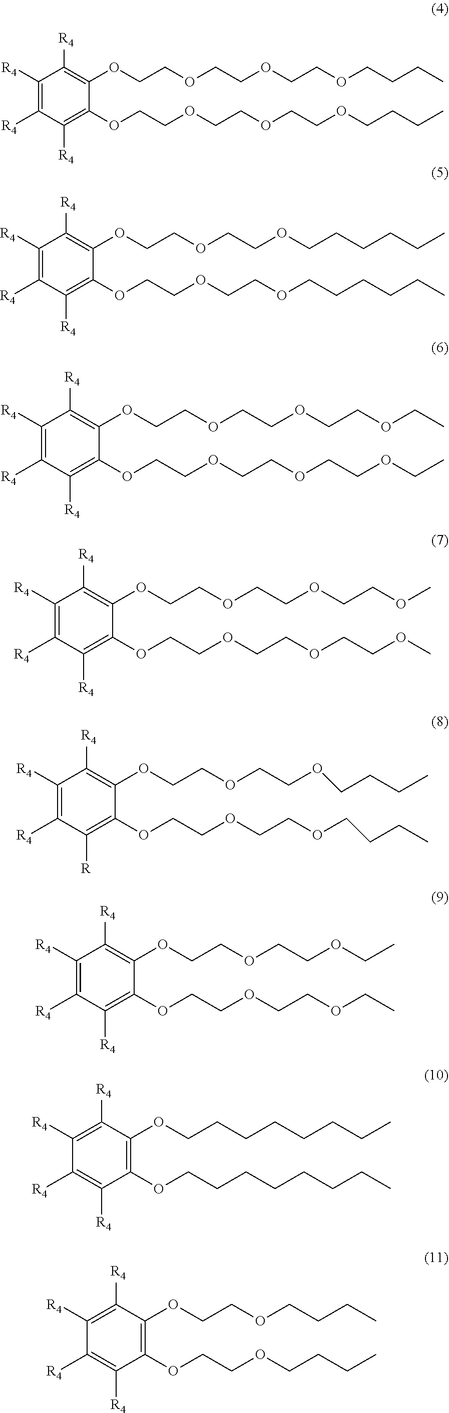

wherein each R₄ is a hydrogen, a sulfonic acid group, or a salt of the sulfonic acid group, and wherein at least one R₄ is a sulfonic acid group or salt of the sulfonic acid group.

An additional example of a suitable surfactant composition may include, but is not limited to, a sulfonated aromatic ether of Formula (12) as follows:

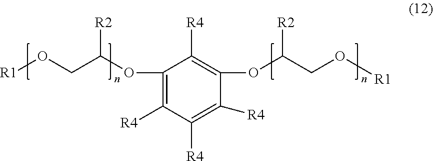

wherein R₁ is a linear or branched alkyl group having from 1 carbon atom to 20 carbon atoms, each R₂ is the same or different and is hydrogen or an alkyl group having from 1 carbon atom to 4 carbon atoms, each R₄ is a hydrogen, a sulfonic acid group, or a salt of the sulfonic acid group, and each n is a value from 0 to 8, wherein at least one R₄ is a sulfonic acid group or a salt of the sulfonic acid group. The alkyl or alkenyl (or heteroatom substituted) groups may be the same or different and, in some embodiments, may include 1 carbon atom to 20 carbon atoms, from 1 carbon atom to 10 carbon atoms, from 1 carbon atom to 8 carbon atoms, or from 1 carbon atom to 4 carbon atoms.

Examples of suitable surfactant compositions of Formula (12) includes those represented by Formulae (13) to (19) further having at least one sulfonic acid group (or salt of the sulfonic acid group) at the 2-position, 4-position, 5-position and/or 6-position:

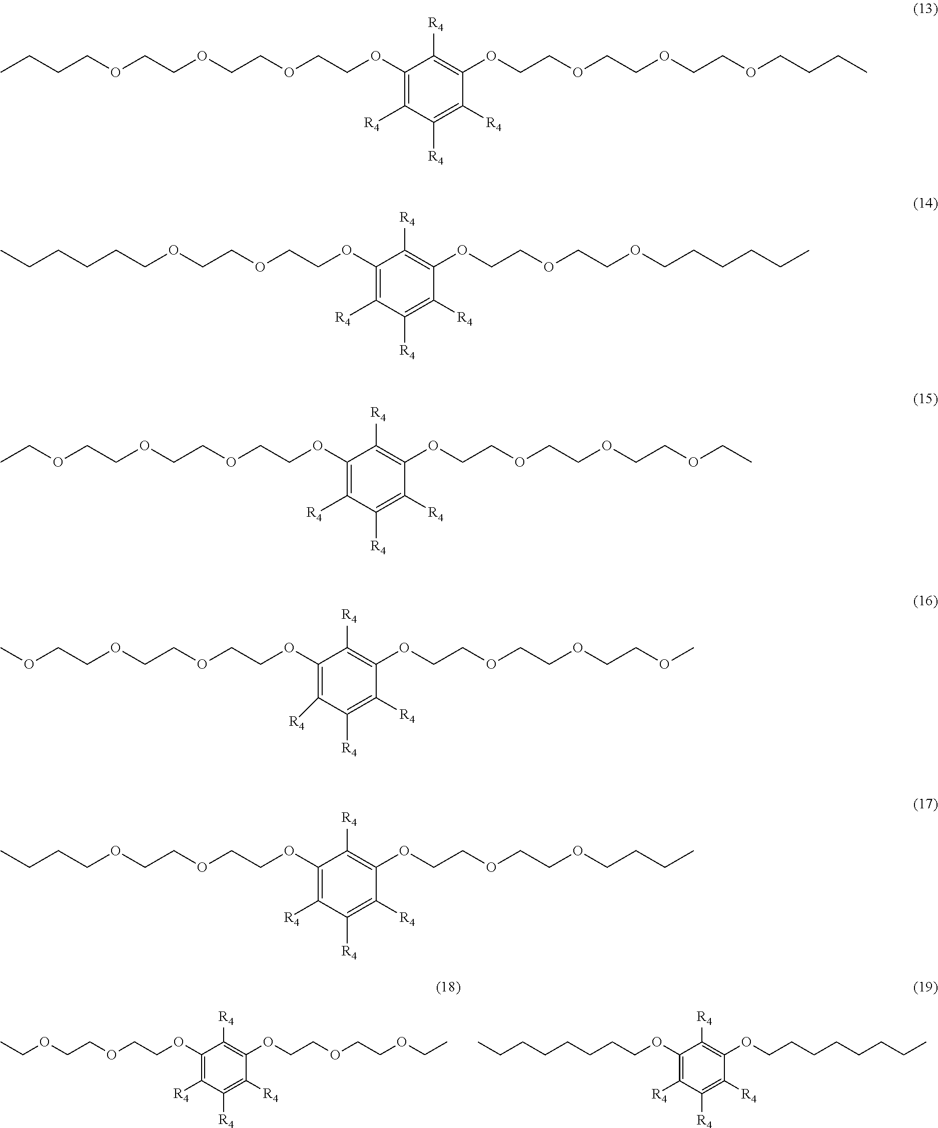

wherein each R₄ is a hydrogen, a sulfonic acid group, or a salt of the sulfonic acid group, and wherein at least one R₄ is a sulfonic acid group or salt of the sulfonic acid group.

An additional example of a suitable surfactant composition may include, but is not limited to, a sulfonated aromatic ether of Formula (20) as follows:

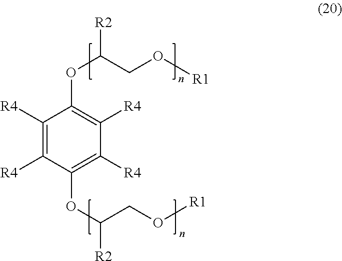

wherein R₁ is a linear or branched alkyl group having from 1 carbon atom to 20 carbon atoms, each R₂ is the same or different and is hydrogen or an alkyl group having from 1 carbon atom to 4 carbon atoms, each R₄ is a hydrogen, a sulfonic acid group, or a salt of the sulfonic acid group, and each n is a value from 0 to 8, wherein at least one R₄ is a sulfonic acid group or a salt of the sulfonic acid group. The alkyl or alkenyl (or heteroatom substituted) groups may be the same or different and, in some embodiments, may include 1 carbon atom to 20 carbon atoms, from 1 carbon atom to 10 carbon atoms, from 1 carbon atom to 8 carbon atoms, or from 1 carbon atom to 4 carbon atoms.

An additional example of a suitable surfactant composition may include, but is not limited to, a sulfonated aromatic ether of Formula (21) as follows:

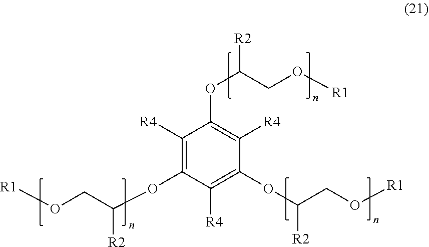

wherein R₁ is a linear or branched alkyl group having from 1 carbon atom to 20 carbon atoms, each R₂ is the same or different and is hydrogen or an alkyl group having from 1 carbon atom to 4 carbon atoms, each R₄ is a hydrogen, a sulfonic acid group, or a salt of the sulfonic acid group, and each n is a value from 0 to 8, wherein at least one R₄ is a sulfonic acid group or a salt of the sulfonic acid group. The alkyl or alkenyl (or heteroatom substituted) groups may be the same or different and, in some embodiments, may include 1 carbon atom to 20 carbon atoms, from 1 carbon atom to 10 carbon atoms, from 1 carbon atom to 8 carbon atoms, or from 1 carbon atom to 4 carbon atoms.

The preceding embodiments of the surfactant compositions shown above with respect to Formulae (2) to (21) are directed to monocyclic aromatic rings. Embodiments of the surfactant compositions may also include bicyclic aromatic rings. An example of a suitable surfactant composition with a bicyclic ring may include, but is not limited to, a sulfonated aromatic ether of Formula (22):

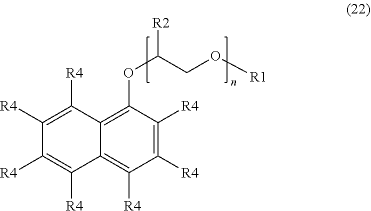

wherein R₁ is a linear or branched alkyl group having from 1 carbon atom to 20 carbon atoms, each R₂ is the same or different and is hydrogen or an alkyl group having from 1 carbon atom to 4 carbon atoms, each R₄ is a hydrogen, a sulfonic acid group, or a salt of the sulfonic acid group, and each n is a value from 0 to 8, wherein at least one R₄ is a sulfonic acid group or a salt of the sulfonic acid group. The alkyl or alkenyl (or heteroatom substituted) groups may be the same or different and, in some embodiments, may include 1 carbon atom to 20 carbon atoms, from 1 carbon atom to 10 carbon atoms, from 1 carbon atom to 8 carbon atoms, or from 1 carbon atom to 4 carbon atoms.

An example of a suitable surfactant composition of Formula (22) with a bicyclic ring may include, but is not limited to, a sulfonated aromatic ether of Formula (23):

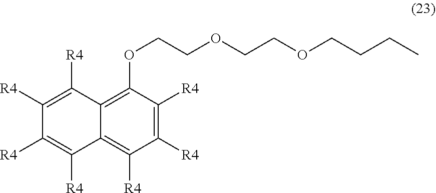

wherein R₁ is a linear or branched alkyl group having from 1 carbon atom to 20 carbon atoms, each R₂ is the same or different and is hydrogen or an alkyl group having from 1 carbon atom to 4 carbon atoms, each R₄ is a hydrogen, a sulfonic acid group, or a salt of the sulfonic acid group, and each n is a value from 0 to 8, wherein at least one R₄ is a sulfonic acid group or a salt of the sulfonic acid group. In at least one embodiment, the R₄ at the 4 position is the sulfonic acid group. The alkyl or alkenyl (or heteroatom substituted) groups may be the same or different and, in some embodiments, may include 1 carbon atom to 20 carbon atoms, from 1 carbon atom to 10 carbon atoms, from 1 carbon atom to 8 carbon atoms, or from 1 carbon atom to 4 carbon atoms.

The embodiments of this disclosure as described herein with respect to the surfactant compositions of Formula (2) apply equally to the compounds of Formula (3) to (23).

As previously described, the surfactant compositions may be multi-functional. The surfactant compositions should also be water-soluble. As the surfactant compositions are produced from aromatic eithers with good lubricant properties, the surfactant compositions may also have properties suitable for use in lubricating oils. For example, the surfactant compositions may have low viscosity and low volatility.

Example surfactant compositions of this disclosure can be prepared by a process that involves reacting an aromatic ether with a sulfonating reagent. The reaction can be carried out in the presence (or absence) of a solvent. The reaction can be carried out under conditions sufficient to produce a surfactant composition represented by any one of Formulae (2) to (23). In some embodiments, the sulfonic acid groups formed sulfonation can be neutralized by reaction with base to form a salt of the sulfonic acid group, for example, a sodium salt of the sulfonic acid.

Suitable aromatic ethers may include an aromatic ring with substituents comprising an ether group represented by Formula (1) below:

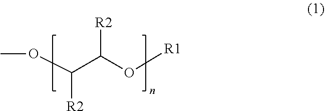

wherein R₁ is a linear or branched alkyl group having from 1 carbon atom to 20 carbon atoms, each R₂ is the same or different and is hydrogen or an alkyl group having from 1 carbon atom to 4 carbon atoms, and n is a value from 0 to 8. Illustrative R₁ substituents include, for example, linear or branched alkyl groups having from 1 carbon atom to 20 carbon atoms, linear or branched alkyl groups having from 1 carbon atom to 10 carbon atoms, or linear or branched alkyl groups having from 1 to 8 carbon atoms. Illustrative R₁ substituents include, for example, methyl, ethyl, propyl, butyl, pentyl, hexyl, heptyl, octyl, and the like. Illustrative R₂ substituents include, for example, hydrogen and alkyl groups having from 1 carbon atom to 4 carbon atoms (e.g., methyl, ethyl, propyl, butyl, and the like). Values of n can range from 0 to 8, from 0 to 6, or from 0 to 4.

The aromatic ring may be a five- or six-membered monocyclic ring or a ten- or twelve-membered bicyclic ring. In some embodiments, the aromatic ring may heterocyclic or carbocyclic. Additional substituents may also be present on the aromatic ring of the sulfonated aromatic ether, including, but not limited to, an alkyl group, an alkenyl group, a heteroatom-substituted alkyl group, a hetero-atom substituted alkenyl group, or a heteroatom, such as F, Cl, or Br. The alkyl or alkenyl (or heteroatom substituted) groups may be the same or different and, in some embodiments, may include 1 carbon atom to 20 carbon atoms, from 1 carbon atom to 10 carbon atoms, from 1 carbon atom to 8 carbon atoms, or from 1 carbon atom to 4 carbon atoms. For example, suitable additional substituents may include methyl, ethyl, ethyl, propyl, butyl, pentyl, hexyl, heptyl, octyl, and the like.

In at least one embodiment, the aromatic ring may be substituted with more than one ether group of Formula (I). The ether groups may be positioned on the aromatic ring at any suitable position. For example, the aromatic ring may have an ether group in the 1-position and one or more additional ether groups at the 2-position, 3-position, 4-position, 5-position, and/or 6-position. By way of further example, the aromatic ring may have ether groups in the 1-position and the 2-position. The ether groups on the aromatic ring may be the same or different.

In at least one embodiment, the aromatic ring may be substituted with one or more sulfonic acid groups. For example, the aromatic ring may include an ether group in the 1-position and one or more sulfonic groups in the 2-position, 3-position, 4-position, 5-position, and/or 6-position. In some embodiments, the aromatic ring may include either groups in the 4-position and the 5-position. A specific example of a suitable sulfonated aromatic ether may include a 6-membered carbocyclic ring with ether groups in the 1-position and 2-position and a sulfonic acid group in the 5-position. Another specific example of a suitable sulfonated aromatic ether may include a 6-member carbocyclic ring with ether groups in the 1-position and 2-position and sulfonic acid groups in the 4-position and the 5-position.

An example of a suitable aromatic ether may include, but is not limited to, an aromatic ether of Formula (24) as follows:

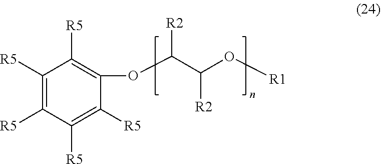

wherein R₁ is a linear or branched alkyl group having from 1 carbon atom to 20 carbon atoms, each R₂ is the same or different and is hydrogen or an alkyl group having from 1 carbon atom to 4 carbon atoms, each R₅ is a hydrogen, an ether group of Formula (I), an alkyl group, an alkenyl group, a heteroatom-substituted alkyl group, a hetero-atom substituted alkenyl group, or a heteroatom, such as F, Cl, or Br, and each n is a value from 0 to 8. The alkyl or alkenyl (or heteroatom substituted) groups may be the same or different and, in some embodiments, may include 1 carbon atom to 20 carbon atoms, from 1 carbon atom to 10 carbon atoms, from 1 carbon atom to 8 carbon atoms, or from 1 carbon atom to 4 carbon atoms.

An additional example of a suitable aromatic ether may include, but is not limited to, an aromatic ether of Formula (25) below:

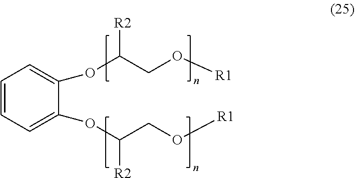

wherein R₁ is a linear or branched alkyl group having from 1 carbon atom to 20 carbon atoms, each R₂ is the same or different and is hydrogen or an alkyl group having from 1 carbon atom to 4 carbon atoms, and each n is a value from 0 to 8.

An additional example of a suitable aromatic ether may include, but is not limited to, an aromatic ether of Formula (26) below:

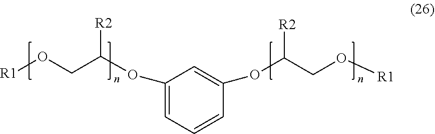

wherein R₁ is a linear or branched alkyl group having from 1 carbon atom to 20 carbon atoms, each R₂ is the same or different and is hydrogen or an alkyl group having from 1 carbon atom to 4 carbon atoms, each R₃ is a hydrogen, an ether group of Formula (I), an alkyl group, an alkenyl group, a heteroatom-substituted alkyl group, a hetero-atom substituted alkenyl group, or a heteroatom, such as F, Cl, or Br, and each n is a value from 0 to 8.

An additional example of a suitable aromatic ether may include, but is not limited to, an aromatic ether of Formula (27) below:

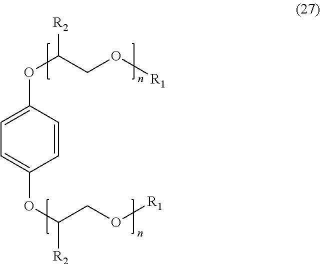

wherein R₁ is a linear or branched alkyl group having from 1 carbon atom to 20 carbon atoms, each R₂ is the same or different and is hydrogen or an alkyl group having from 1 carbon atom to 4 carbon atoms, each R₃ is a hydrogen, an ether group of Formula (I), an alkyl group, an alkenyl group, a heteroatom-substituted alkyl group, a hetero-atom substituted alkenyl group, or a heteroatom, such as F, Cl, or Br, and each n is a value from 0 to 8.

An additional example of a suitable aromatic ether may include, but is not limited to, an aromatic ether of Formula (28) below:

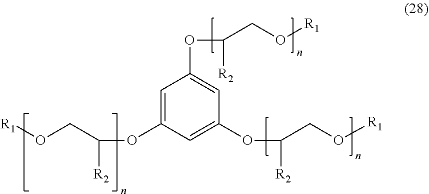

wherein R₁ is a linear or branched alkyl group having from 1 carbon atom to 20 carbon atoms, each R₂ is the same or different and is hydrogen or an alkyl group having from 1 carbon atom to 4 carbon atoms, each R₃ is a hydrogen, an ether group of Formula (I), an alkyl group, an alkenyl group, a heteroatom-substituted alkyl group, a hetero-atom substituted alkenyl group, or a heteroatom, such as F, Cl, or Br, and each n is a value from 0 to 8.

Any of a variety of suitable sulfonating reagents capable for reaction with the aryl ether to add a sulfonic acid group to the aryl ether may be used. Examples of suitable sulfonate reagents may include, but are not limited to, silica-sulfuric acid, sulfuric acid solutions (e.g., concentrated sulfuric acid), sulfur trioxide-dioxane complexes, trifluoro acetic acid, and sulfur trioxide in dichloromethane. In at least one embodiment, the sulfonating reagent may include silica-sulfuric acid. Silica-sulfuric acid is a solid acid and may be prepared, for example, by reaction of silica with a sulfuric acid (e.g., chlorosulfonic acid). The silica-sulfuric acid may be an advantageous sulfonating reagent, for example, as the silica-sulfuric acid can have short reaction times and high yields without formation of sulfones as by-products.

Other example reaction ingredients include, but are not limited to, solvents, such as toluene, xylene, decane, heptane, and dichloro alkanes. Suitable dichloro alkanes may have, for example, from 1 carbon atom to 6 carbon atoms. In at least one embodiment, the solvent may include dichloromethane.

Reaction conditions for reaction of the aromatic ether with the sulfonating regent, such as temperature, pressure and contact time, may vary greatly and any suitable combination of such conditions may be employed herein. The reaction temperature may range, for example, between 25° C. to 250° C., from 30° C. to 200° C., or from 60° C. to 150° C. Normally the reaction is carried out under ambient pressure and the contact time may vary from a matter of seconds or minutes to a few hours or greater. The reactants can be added to the reaction mixture or combined in any order. The stir time employed can range, for example, from 0.5 hour to 96 hours, from 12 hours to 72 hours, or from 24 hours to 60 hours.

Accordingly, the preceding description describes surfactants formed from sulfonation of aromatic ethers. The compositions and methods disclosed herein may include any of the various compositions and methods disclosed herein, including one or more of the following embodiments.

### Embodiment 1

The example surfactant composition may include a sulfonated aromatic ether. The sulfonated aromatic ether may include an aromatic ring with substituents comprising an ether group and a sulfonic acid group or a salt of the sulfonic acid group. The ether group may be represented by Formula (1).

### Embodiment 2

The surfactant composition of embodiment 1, wherein the aromatic ring is a six-membered monocyclic ring.

### Embodiment 3

The surfactant composition of embodiment 1 or 2, wherein the aromatic ring is a six-membered monocyclic ring with the ether group at the 1-position, the ether group at the 2-position, or the either group at the 1-position with an additional ether group at the 2-position.

### Embodiment 4

The surfactant composition of embodiment 1 or 2, wherein the aromatic ring is a six-membered monocyclic ring with the ether group at the 1-position and with the sulfonic acid group or the salt of the sulfonic acid group at any unsubstituted position on the six-member monocyclic ring.

### Embodiment 6

The surfactant composition of embodiment 1 or 2, wherein the sulfonated aromatic ether is represented by Formula (2).

### Embodiment 7

The surfactant composition of embodiment 1 or 2, wherein the sulfonated aromatic ether is represented by Formula (3).

### Embodiment 7

The surfactant composition of embodiment 1 or 2, wherein the sulfonated aromatic ether is represented by at least one of formulae (4) to (11) further having the sulfonic acid group or the salt of the sulfonic acid group at any one of the 3-position, the 4-position, 5-position and/or 6-position.

### Embodiment 8

The surfactant composition of embodiment 1 or 2, wherein the sulfonated aromatic ether is represented by Formula (12).

### Embodiment 9

The surfactant composition of embodiment 1 or 2, wherein the sulfonated aromatic ether is represented by at least one of formulae (13) to (19) further having the sulfonic acid group or the salt of the sulfonic acid group at the 2-position, 4-position, 5-postion and/or 6-position.

### Embodiment 10

The surfactant composition of embodiment 1 or 2, wherein the sulfonated aromatic ether is represented by Formula (20).

### Embodiment 11

The surfactant composition of embodiment 1 or 2, wherein the sulfonated aromatic ether is represented by Formula (21).

### Embodiment 12

The surfactant composition of embodiment 1, wherein the aromatic ring is a 10-membered or 12-membered bicyclic ring.

### Embodiment 13

The surfactant composition of embodiment 1 or 12, wherein the sulfonated aromatic ether is represented by Formula (22).

### Embodiment 14

A method for producing a surfactant composition. The example method may comprise reacting an aromatic ether with a sulfonating reagent to form the surfactant composition, wherein the aromatic ether comprises an ether group represented by Formula (I). The surfactant composition may comprise a sulfonated aromatic ether comprising an aromatic ring with substituents comprising the ether group and a sulfonic acid group.

### Embodiment 15

The method of embodiment 14, herein the reacting occurs at a reaction temperature from about 60° C. to about 150° C.

### Embodiment 16

The method of embodiment 14 or 15, wherein the reacting occurs in the presence of a dichloro alkane.

### Embodiment 17

The method of any one of embodiments 14 to 16, wherein the sulfonating reagent comprises a silica-sulfuric acid.

### Embodiment 18

The method of any one of embodiments 14 to 17, wherein the aromatic ether is represented by Formula 25.

### Embodiment 19

The method of any one of embodiments 14 to 17, wherein the aromatic ether is represented by Formula 26.

### Embodiment 20

The method of any one of embodiments 14 to 17, wherein the aromatic ether is represented by Formula 27.

### Embodiment 21

The method of any one of embodiments 14 to 17, wherein the aromatic ether is represented by Formula 28.

### Embodiment 22

The method of any one of embodiments 14 to 17, wherein the aromatic ether is represented by Formula 29.

### Embodiment 23

The method of any one of embodiments 14 to 17, wherein the aromatic ether comprises a 10-member or 12-member bicyclic ring.

### Embodiment 24

The method of any one of embodiments 14 to 23, further comprising neutralizing the sulfonic acid group to form a salt of the sulfonic acid group.

### EXAMPLES

To facilitate a better understanding of the embodiments described herein, the following examples of certain aspects of some embodiments are given. In no way should the following examples be read to limit, or define, the scope of the present disclosure.

### Example 1

### Synthesis of 1,2-bis(2-(2-hexloxy)ethoxy)ethoxy)benzene

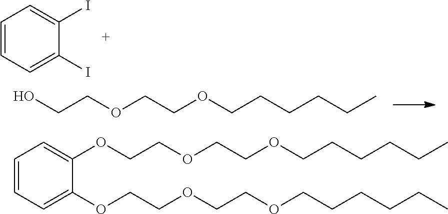

Copper (I) iodide (FW 190.45, 1.80 grams, 9.45 mmol), 1,10-phenathraoline (FW 180.23, 3.30 grams, 18.30 mmol), cesium carbonate (FW 325.82, 38.00 grams, 116.60 mmol), 1,2-diiodobenzene (FW 329.90, 15.00 grams, 45.50 mmol), di(ethylene glycol) hexyl ether (FW 190.28, 26.00 grams, 136.60 mmol) and 100 milliliters of dry xylene were charged in a 500 ml round bottom flask. The reaction mixture was heated with stirring at 140° C.2 for 72 hours under nitrogen. The resulting suspension was cooled to room temperature and filtered through celite and alumina. The filtrate was concentrated at high vacuum. The residue was purified by flask chromatography on silica gel with 1:1 hexane and ethyl acetate. The yield of the final dark yellow product was 17.00 grams (85%). The product IR, and NMR analysis shows the formation of aryl ether product. IR: neat (cm⁻¹): 3064, 2929, 2829, 1594, 1501, 1455, 1378, 1353, 1328, 1256, 1221, 1123, 1056, 931, 743. ¹H NMR (CDCl₃): δ 6.75 (m, 4H, Ph), 4.02 (t, 4H, —OCH₂—), 3.71 (m, 4H, —CH₂O—) 3.56 (m, 4H, —OCH2), 3.44 (m, 4H, —CH₂O—,), 3.30 (t, 4H, —OCH₂—), 1.43-1.13 (m 16H, —CH₂—), 0.73 (t, 6H, —CH₃). ¹³CNMR (CDCl₃): 149.2, 121.8, 115.1, 71.5, 70.9, 70.2, 69.8, 69.0, 31.7, 29.7, 25.8, 22.7, 14.0. GCMS spectrum of mono di(ethylene glycol) hexyl-aryl ether showed m/e at 266.1928. The exact mass for C₁₆H₂₆O₃ would be 266.19. Differential scanning calorimetry (DSC) of the aryl ether of this Example 1 showed no crystallization peak with single low temperature Tg of −82.5° C. suggesting the fluid would have excellent low temperature properties.

### Example 2

### Lubricant Properties

The fluid prepared in Example 1 was evaluated as a synthetic basestock and found to have good lubricant properties. The kinematic viscosity (Kv) of the liquid product was measured using ASTM standard D-445 and reported at temperatures of 100° C. (Kv at 100° C.) or 40° C. (Kv at 40° C.). The viscosity index (VI) was measured according to ASTM standard D-2270 using the measured kinematic viscosities for the product. Thermogravimetric analysis (TGA) was measured according to standard procedure. The lube properties of the product of Example 1 was evaluated and the data are shown in Table 1 below along with data for a common polyalphaolefin (PAO4) lubricant. TGA Noack can be used as Noack surrogate and for the fluid of the Example 1, it was found to be 8.3 which is lower than TGA Noack of 13.45 for PAO4.

Table 1 below summarizes the lubricant properties of the 1,2-bis(2-(2-hexloxy)ethoxy)ethoxy)benzene prepared in Example 1 against those of PAO4.

TABLE 1

| | Kinematic | Kinematic | Viscosity | |
|---|---|---|---|---|
| Basestock | Viscosity at | Viscosity at | Index | TGA |
| # | 100° C. (Kv₁₀₀) | 40° C. (Kv₄₀) | (VI) | (50% wt. loss) |
| Example 1 | 3.14 | 13.3 | 94 | 289.5 |
| PAO4 | 4.10 | 19.00 | 126 | 259.0 |

The fluid of the Example 1 has lower viscosity than PAO4 (Kv₁₀₀ 3.14 vs. 4.1) but has higher stability (289.5° C. 50% wt. loss vs. 259° C.) than PAO4.

### Example 3

### Sulfonation of 1,2-bis(2-(2-hexloxy)ethoxy)ethoxy)benzene

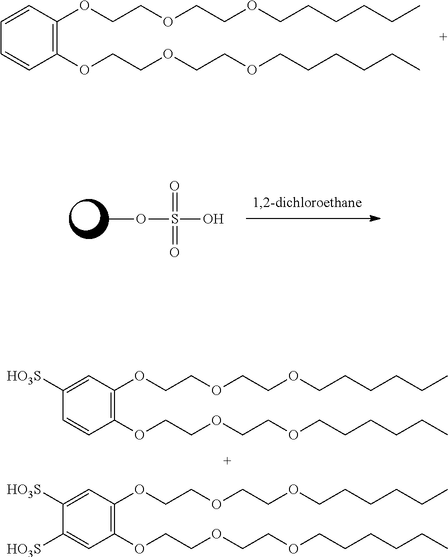

A 100 ml round bottomed flask was charged with 44% silica sulfuric acid (4.78 g, 0.02145 mol, 1.5 eq) and 1,2-bis(2-(2-(hexyloxy)ethoxy)ethoxy)benzene (5 g, 0.0110 mole, 1 eq) in 15 ml of 1,2-dichloro ethane. The reaction mixture was stirred at 80° C. for 48 hours. The heterogeneous mixture then filtered, washed with 50 ml of dichloromethane and 50 ml of methanol. The solvent was removed under reduced pressure. The residue was washed with hexane (2×20 ml) to remove the unreacted starting material. The dried isolated product was characterized by ¹H NMR and FTICR Mass spectrometry. The product ¹H NMR at 7.8 ppm and molecular ion peak at 533.27 indicates the presence of sulfonation of 1,2-bis(2-(2-(hexyloxy)ethoxy) ethoxy)benzene. APPI mass spectrometry indicates that the product comprised an approximately 50:50 mix of monosulfonated and disulfonated components.

### Example 4

### Surface Tension Measurements of Sulfonated 1,2-bis(2-(2-hexloxy)ethoxy)ethoxy)benzene

To determine the surfactant properties of the sulfonated 1,2-bis(2-(2-hexloxy)ethoxy)ethoxy)benzene prepared in Example 4, surface tension measurements were made. Surface tension measurements were made using the Wilhelmy plate apparatus for the sulfonated 1,2-bis(2-(2-(hexyloxy)ethoxy)ethoxy)benzene dissolved in aqueous 0.1% w/w Na2CO3.

Table 2 below shows the results of the measured surface tension versus concentration of the sulfonated 1,2-bis(2-(2-(hexyloxy)ethoxy)ethoxy)benzene in the aqueous 0.1% w/w Na2CO3 solution. For comparison, the surface tension of water is 72 mN/m.

TABLE 2

| | Surfactant | Surface |
|---|---|---|
| | Concentration | Tension |
| Entry | (mM) | (mN/m) |
| 1 | 0.011 | 56.5 |
| 2 | 0.034 | 44.9 |
| 3 | 0.107 | 41.1 |
| 4 | 0.340 | 36.7 |
| 5 | 1.076 | 33.8 |
| 6 | 3.401 | 32.1 |
| 7 | 6.048 | 31.1 |
| 8 | 9.125 | 29.3 |

The surface tension versus concentration shown in Table 2 above does not reach a constant value above a certain concentration (designated as the critical micelle concentration, indicating micelle formation) with increase in concentration, due to the pH variation with increase in concentration. However, micelle formation was verified, and critical micelle concentration was estimated through fluorescence measurements using pyrene as the fluorescence probe at an excitation wavelength of 335 nm. The intensity ratio of the 1st peak (occurring at approximately 373 nm) to the 3rd peak (occurring at approximately 383 nm) in the fluorescence spectrum of pyrene, denoted by I1/I3, is known to drop when the pyrene transitions into a hydrophobic environment from a hydrophilic environment. This transition occurs above the critical micelle concentration, when the pyrene partitions inside the hydrophobic micelle cores.

Table 3 shows I1/I3 as a function of concentration for sulfonated 1,2-bis(2-(2-(hexyloxy)ethoxy)ethoxy)benzene dissolved in aqueous 0.1% w/w Na2CO3.

TABLE 3

| | Surfactant | |
|---|---|---|
| Entry | Concentration (mM) | I₁/I₃ |
| 1 | 0.010 | 1.776 |
| 2 | 0.032 | 1.779 |
| 3 | 0.108 | 1.787 |
| 4 | 0.322 | 1.698 |
| 5 | 0.592 | 1.452 |
| 6 | 1.077 | 1.338 |
| 7 | 3.223 | 1.200 |
| 8 | 5.365 | 1.107 |
| 9 | 10.728 | 0.925 |

From Table 3, it can be observed that the transition of pyrene into a hydrophobic environment from a hydrophilic environment occurs at about 0.32 mM, indicating the formation of micelles. This critical micelle concentration is lower than that of many commercial surfactants; for example, the critical micelle concentration of sodium dodecyl sulfate is 8 mM. Lower critical micelle concentration is desirable in applications to reduce surfactant consumption.

As previously mentioned, pH of the sulfonated 1,2-bis(2-(2-(hexyloxy)ethoxy)ethoxy)benzene in the aqueous 0.1% w/w Na2CO3 solution varies with concentration. Table 4 below shows the pH versus surfactant concentration of the sulfonated 1,2-bis(2-(2-(hexyloxy)ethoxy)ethoxy)benzene in the aqueous 0.1% w/w Na2CO3 solution.

TABLE 4

| | Surfactant | |
|---|---|---|
| Entry | Concentration (mM) | pH |
| 1 | 0.0107 | 11 |
| 2 | 0.1070 | 10.95 |
| 3 | 0.3209 | 10.88 |
| 4 | 1.0708 | 10.58 |
| 5 | 3.3381 | 8.92 |
| 6 | 5.5736 | 7 |
| 7 | 11.1473 | 2.11 |

Due to the sulfonic acid groups present in sulfonated 1,2-bis(2-(2-(hexyloxy)ethoxy)ethoxy)benzene, the pH decreases with increase in concentration. However, as shown in Table 4 above, the pH of sulfonated 1,2-bis(2-(2-(hexyloxy)ethoxy)ethoxy)benzene dissolved in 0.1% w/w Na2CO3 remains at or above 7 at a concentration of 5 mM and below, demonstrating that surface tension reduction can be achieved at the concentration and pH ranges viable for commercial applications.

### Example 5

### Synthesis of 1,2-bis(2-(2-butoxy)ethoxy)ethoxy)benzene

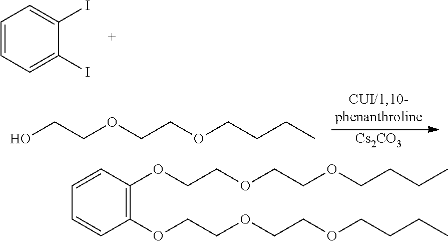

1,2-diiodobenzene (5 g, 0.0152 mol, MW:329.91), di(ethylene glycol)butyl ether (9.83 g, 0.0606 mol, MW:162.23), cesium carbonate, (12.44 g, 0.0379 mol, MW:328.5), 1,10-phenathroline (0.546 g, 0.00303 mol, MW:180.21), copper (I) iodide (0.0.60 g, 0.0030 mol, MW:195.01) and 50 ml of dry xylene were charged in 350 ml three necked round bottom flask. The reaction mixture was heated with stirring at 140° C. for 24 hours under nitrogen. The resulting suspension was cooled to room temperature and filtered through celite. The filtrate was concentrated at 180° C. under high vacuum. The residue was purified by flask chromatography on silica gel with hexane. The yield of the final pale-yellow product was 4.0 grams. The product ¹³C NMR analysis suggests the formation of 1,2-diarylether glycol ether product. ¹³C NMR (CDCl₃): 149.19, 121.89, 115.13, 71.14, 70.87, 70.16, 69.79, 68.95, 31.62, 19.46, 13.78

### Example 6

### Sulfonation of 1,2-bis(2-(2-butoxy)ethoxy)ethoxy)benzene

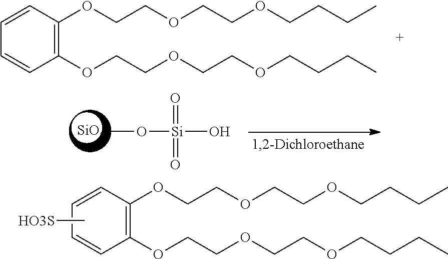

A 100 ml round bottomed flask was charged with 44% silica sulfuric acid (8.94 g, 0.0401, 4.0) and 2-bis(2-(2-butoxyethoxy)ethoxy)benzene (4 g, 0.0100 mole, 1 eq) in 1,2-dichloro ethane (15 ml). The 2-bis(2-(2-butoxyethoxy)ethoxy)benzene was from Example 4. The reaction mixture was stirred at 93° C. for 18 hours. The heterogeneous mixture then filtered, washed with 30 ml of dichloromethane and 30 ml of methanol. The solvent was removed under pressure. The residue was washed with hexane (2×20 ml) to remove unreacted 1,2-bis(2-(2-butoxyethoxy)ethoxy)benzene. The product was completely soluble in the water.

### Example 7

### Synthesis of Sodium Salt of Sulfonated 1,2-bis(2-(2-butoxy)ethoxy)ethoxy)benzene

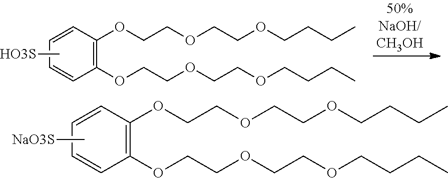

3 g of Sulfonated of 1,2-bis(2-(2-butoxyethoxy)ethoxy)benzene from Example 5 was dissolved in 10 ml of methanol (pH ˜4.0) at RT. Next, 50% aqueous NaOH (2.5 g) solution was slowly added to precipitate the pale-yellow sodium salt of 1,2-bis(2-(2-butoxyethoxy)ethoxy)benzene. The product was then filtered and washed with 15 ml of methanol. The product was dried overnight at 60° C. in a vacuum oven. The isolated product was characterized by IR. The product IR peaks at 1622, 1575, 1494, 1128, 782 indicates the presence sodium salt of sulfonated 1,2-bis(2-(2-butoxy)ethoxy)ethoxy)benzene. The product was soluble in water.

### Example 8

### Synthesis of 1-(2-(2-butoxyethoxy)ethoxy) naphthalene

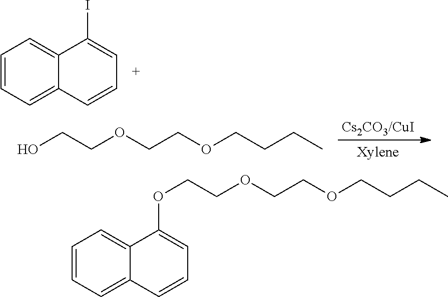

1-iodonapthalene (10 g, 39.359 mmol, mw:254.07), di(ethylene glycol)butyl ether (12.78 g, 78.70 mmol, mw:162.23), cesium carbonate, (19.40 g, 59.10 mmol, mw:328.5), 1,10-phenathroline (1.408 g, 7.873 mmol, mw:180.21), copper (I) iodide (0.768 g, 3.90 mmol, mw:195.01) and 70 ml of dry xylene where charged into 350 ml three necked round bottom flask. The reaction mixture was heated with stirring at 145° C. for 26 hours under nitrogen. The resulting suspension was cooled to room temperature and filtered through celite. The filtrate was concentrated at 180° C. under high vacuum. The residue was purified by flask chromatography on silica gel with hexane. The yield of the final pale-yellow product was 9.0 grams (79%). The product ¹³C NMR analysis suggests the formation of naphthyl glycol ether product. ¹³C NMR (CDCl₃): 154.71, 134.64, 127.51, 126.44, 125.88, 125.80, 125.16, 122.26, 120.43, 104.94, 71.32, 71.08, 70.25, 69.85, 67.94, 31.81, 19.33, 13.97. ¹H NMR (CDCl₃): δ 6.75 (m, 7H, naphthyl), 4.19 (t, 2H, —OCH₂—), 3.91 (m, 2H, —CH₂O—) 3.72 (m, 2H, —OCH₂—), 3.56 (m, 2H, —CH₂O—,), 3.43 (t, 2H, —OCH₂—), 1.43-1.13 (m 4H, —CH₂—), 0.73 (t, 3H, —CH₃).

### Example 9

### Lubricant Properties

The fluid prepared in Example 8 was evaluated as a synthetic basestock and found to have good lubricant properties. The kinematic viscosity (Kv) of the liquid product was measured using ASTM standard D-445 and reported at temperatures of 100° C. (Kv at 100° C.) or 40° C. (Kv at 40° C.). The viscosity index (VI) was measured according to ASTM standard D-2270 using the measured kinematic viscosities for the product. Thermogravimetric analysis (TGA) was measured according to standard procedure. The lube properties of the product of Example 8 was evaluated and the data are shown in Table 5.

TABLE 5

| | Kinematic | | | | |
|---|---|---|---|---|---|
| | Viscosity | Kinematic | Viscosity | TGA | Density |
| Basestock | at 100° C. | Viscosity at | Index | (50% wt. | (40° C.) |
| # | (Kv₁₀₀) | 40° C. (Kv₄₀) | (VI) | loss) | (g/cm³) |
| Example 8 | 2.59 | 11.5 | 26 | 22.3 | 1.041 |

### Example 10

### Sulfonation of 1-(2-(2-butoxyethoxy)ethoxy) naphthalene with silica sulfuric acid

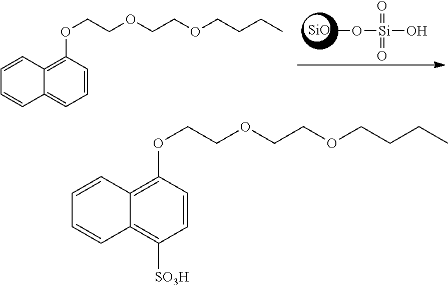

A 100 ml round bottomed flask was charged with 44% silica sulfuric acid (17.0 g, 0.0694 mole, 4.0 equivalent) and 1-(2-(2-butoxyethoxy)ethoxy naphthalene (5 g, 0.01735 mole, 1 eq) in 1,2-dichloro ethane (15 ml). The reaction mixture was stirred at 90° C. for 18 hours. The heterogeneous mixture then filtered and washed with 30 ml of dichloromethane and 50 ml of methanol. The solvent was removed under pressure. The residue was washed with hexane (2×20 ml) to remove unreacted 1-(2-(2-butoxyethoxy)ethoxy naphthalene. The isolated product was characterized by ¹³CNMR: The product ¹³C NMR at 145 peak indicates the presence of sulfonation of 1-(2-(2-butoxyethoxy)ethoxy) naphthalene. The product was dissolved completely in the water.

### Example 11

### Synthesis of Sodium Salt of Sulfonated of 1-(2-(2-butoxyethoxy)ethoxy naphthalene

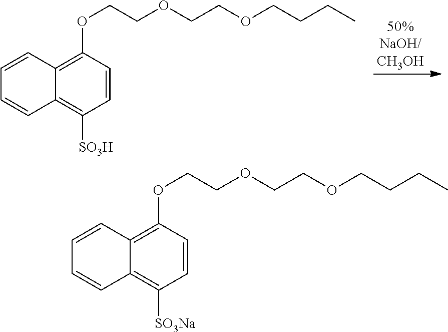

3 grams of sulfonated of 1-(2-(2-butoxyethoxy)ethoxy naphthalene from Example 10 was dissolved in 15 ml of methanol (pH ˜4.0) at 10-15° C. Next, 50% aqueous NaOH (2.0 g) solution was slowly added to precipitate the pale-yellow sodium salt of Sulfonated of 1-(2-(2-butoxyethoxy)ethoxy naphthalene (pH ˜7.0-8.0). The product was then filtered and washed with 15 ml of methanol. The product was dried overnight at 60° C. in a vacuum oven. The isolated product was characterized by NMR. ¹³C NMR of the product showed peaks at 160, 143, 134, 132, 129, 128, 127 and 123 ppm indicating naphthalene ring and peaks at 75 and 63 ppm indicating the —O—CH₂—CH₂—O— group of sulfonation sodium salt of 1-(2-(2-butoxyethoxy)ethoxy) naphthalene. The product was dissolved in water.

While the invention has been described with respect to a number of embodiments and examples, those skilled in the art, having benefit of this disclosure, will appreciate that other embodiments can be devised which do not depart from the scope and spirit of the invention as disclosed herein. Although individual embodiments are discussed, the invention covers all combinations of all those embodiments.

While compositions, methods, and processes are described herein in terms of “comprising,” “containing,” “having,” or “including” various components or steps, the compositions and methods can also “consist essentially of” or “consist of” the various components and steps. The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of the invention, additionally, they do not exclude impurities and variances normally associated with the elements and materials used.

All numerical values within the detailed description and the claims herein modified by “about” or “approximately” with respect the indicated value are intended to take into account experimental error and variations that would be expected by a person having ordinary skill in the art.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited.

1. A surfactant composition comprising:
a sulfonated aromatic ether comprising an aromatic ring with substituents comprising an ether group and a sulfonic acid group or a salt of the sulfonic acid group, wherein the ether group is represented by the following formula: 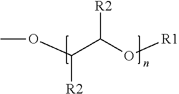
wherein R₁ is a linear or branched alkyl group having from 1 carbon atom to 20 carbon atoms;
wherein each R₂ is individually a hydrogen or an alkyl group having from 1 carbon atom to 4 carbon atoms; and
wherein n is a value from 0 to 8.

2. The surfactant composition of claim 1, wherein the aromatic ring is a six-membered monocyclic ring.

3. The surfactant composition of claim 1, wherein the aromatic ring is a six-membered monocyclic ring with the ether group at the 1-position, the ether group at the 2-position, or the either group at the 1-position with an additional ether group at the 2-position.

4. The surfactant composition of claim 1, wherein the aromatic ring is a six-membered monocyclic ring with the ether group at the 1-position and with the sulfonic acid group or the salt of the sulfonic acid group at any unsubstituted position on the six-member monocyclic ring.

5. The surfactant composition of claim 1, wherein the sulfonated aromatic ether is represented by the following formula: 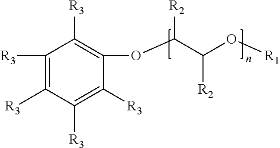
wherein each R₃ is individually a hydrogen, the sulfonic acid group, the salt of the sulfonic acid group, an ether group of Formula (I), an alkyl group, an alkenyl group, a heteroatom-substituted alkyl group, a hetero-atom substituted alkenyl group, or a heteroatom; and
wherein at least one R₃ is the sulfonic acid group or the salt of the sulfonic acid group.

6. The surfactant composition of claim 1, wherein the sulfonated aromatic ether is represented by the following formula: 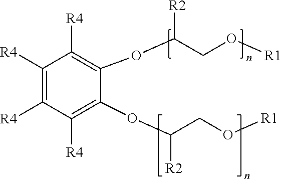
wherein each R₄ is individually a hydrogen, the sulfonic acid group, or the salt of the sulfonic acid group; and
wherein at least one R₄ is the sulfonic acid group or the salt of the sulfonic acid group.

7. The surfactant composition of claim 1, wherein the sulfonated aromatic ether is represented by at least one of the following formulae further having the sulfonic acid group or the salt of the sulfonic acid group at any one of the 3-position, the 4-position, 5-position and/or 6-position: 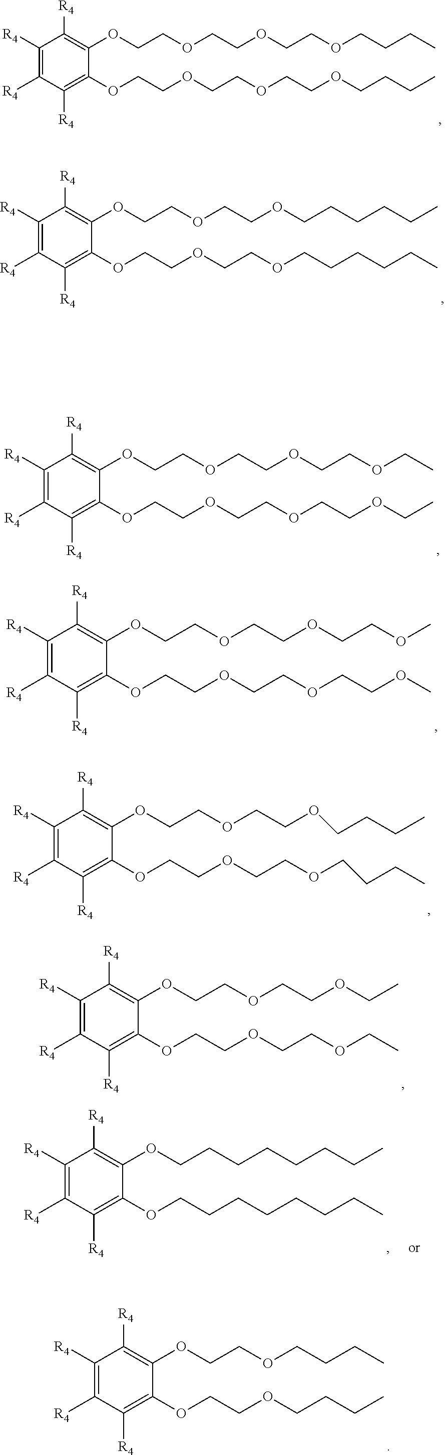
wherein each R₄ is individually a hydrogen, the sulfonic acid group, or the salt of the sulfonic acid group; and
wherein at least one R₄ is the sulfonic acid group or the salt of the sulfonic acid group.

8. The surfactant composition of claim 1, wherein the sulfonated aromatic ether is represented by the following formula: 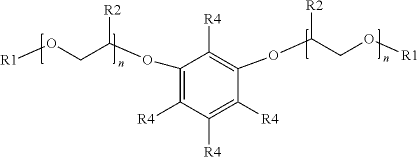
wherein each R₄ is individually a hydrogen, the sulfonic acid group, or the salt of the sulfonic acid group; and
wherein at least one R₄ is the sulfonic acid group or the salt of the sulfonic acid group.

9. The surfactant composition of claim 1, wherein the sulfonated aromatic ether is represented by at least one of the following formulae further having the sulfonic acid group or the salt of the sulfonic acid group at the 2-position, 4-position, 5-position and/or 6-position: 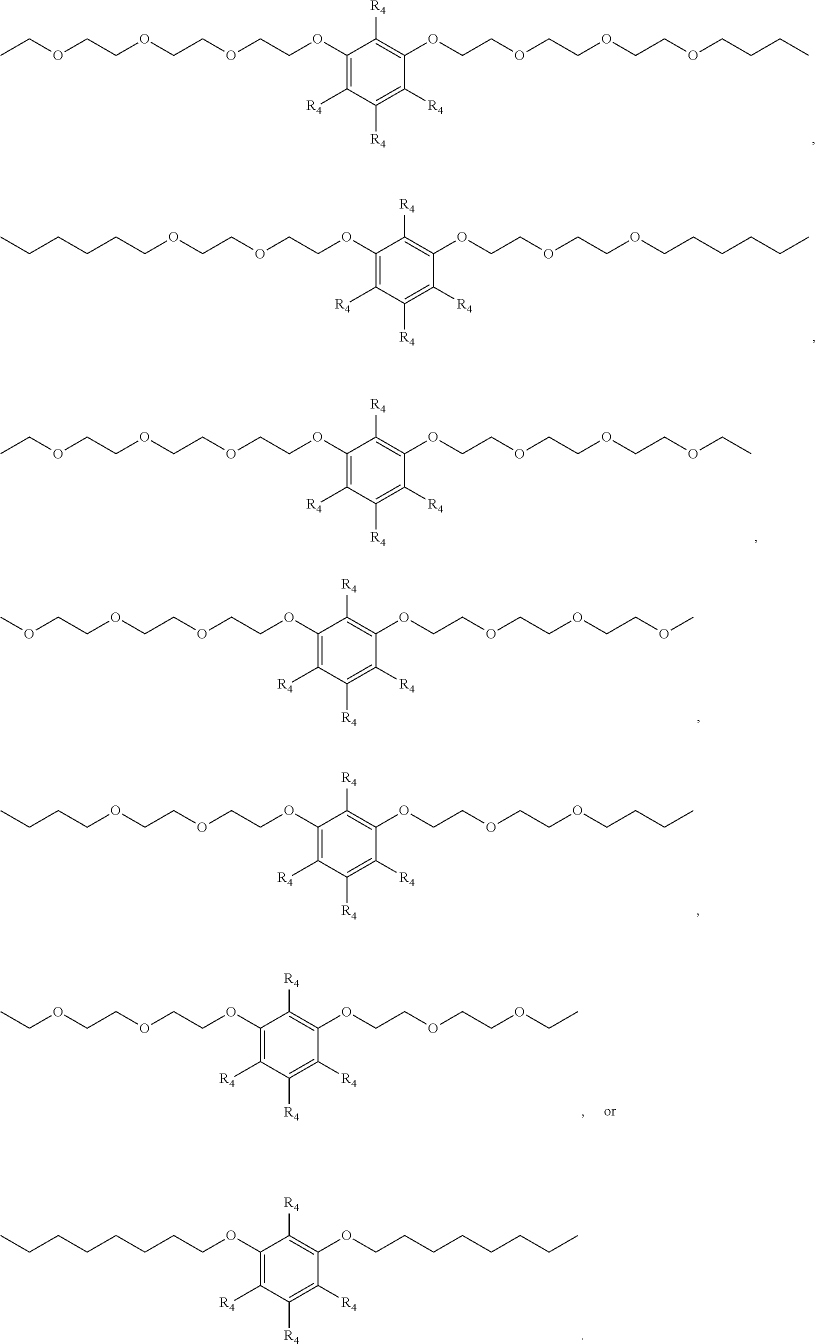
wherein each R₄ is individually a hydrogen, the sulfonic acid group, or the salt of the sulfonic acid group; and
wherein at least one R₄ is the sulfonic acid group or the salt of the sulfonic acid group.

10. The surfactant composition of claim 1, wherein the sulfonated aromatic ether is represented by the following formula: 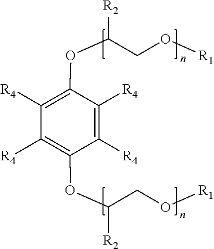
wherein each R₄ is individually a hydrogen, the sulfonic acid group, or the salt of the sulfonic acid group; and
wherein at least one R₄ is the sulfonic acid group or the salt of the sulfonic acid group.

11. The surfactant composition of claim 1, wherein the sulfonated aromatic ether is represented by the following formula: 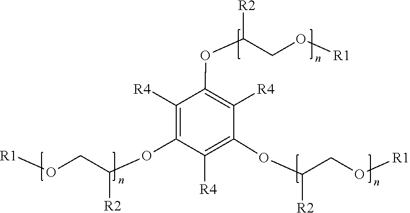
wherein each R₄ is individually a hydrogen or the sulfonic acid group; and
wherein at least one R₄ is the sulfonic acid group.

12. The surfactant composition of claim 1, wherein the aromatic ring is a 10-membered or 12-membered bicyclic ring.

13. The surfactant composition of claim 1, wherein the sulfonated aromatic ether is represented by the following formula: 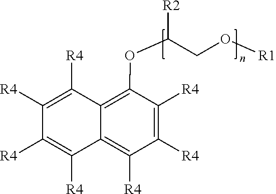
wherein each R₄ is individually a hydrogen, the sulfonic acid group, or the salt of the sulfonic acid group; and
wherein at least one R₄ is the sulfonic acid group or the salt of the sulfonic acid group.

14. A method of forming a surfactant composition comprising:
reacting an aromatic ether with a sulfonating reagent to form the surfactant composition, wherein the aromatic ether comprises an ether group represented by the following formula: 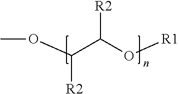
wherein R₁ is a linear or branched alkyl group having from 1 carbon atom to 20 carbon atoms;
wherein each R₂ is individually a hydrogen or an alkyl group having from 1 carbon atom to 4 carbon atoms; and
wherein n is a value from 0 to 8, and
wherein the surfactant composition comprises a sulfonated aromatic ether comprising an aromatic ring with substituents comprising the ether group and a sulfonic acid group.

15. The method of claim 14, wherein the reacting occurs at a reaction temperature from about 60° C. to about 150° C.

16. The method of claim 14, wherein the reacting occurs in the presence of a dichloro alkane.

17. The method of claim 14, wherein the sulfonating reagent comprises a silica-sulfuric acid.

18. The method of claim 14 wherein the aromatic ether is represented by the following formula: 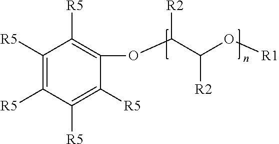
wherein each R₅ is a hydrogen, an ether group of Formula (I), an alkyl group, an alkenyl group, a heteroatom-substituted alkyl group, a hetero-atom substituted alkenyl group, or a heteroatom.

19. The method of claim 14, wherein the aromatic ether is represented by the following formula:

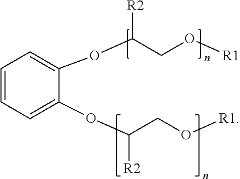

20. The method of claim 14, wherein the aromatic ether is represented by the following formula:

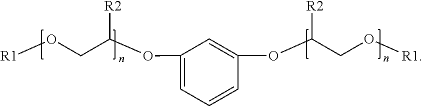

21. The method of claim 14, wherein the aromatic ether is represented by the following formula:

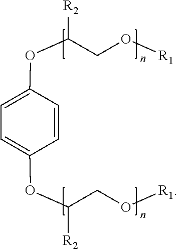

22. The method of claim 14, wherein the aromatic ether is represented by the following formula:

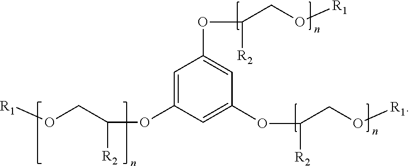

23. The method of claim 14, wherein the aromatic ether comprises a 10-member or 12-member bicyclic ring.

24. The method of claim 14, further comprising neutralizing the sulfonic acid group to form a salt of the sulfonic acid group.
